(19) United States
(12) Patent Application Publication
(10) Pub. No.: US 2024/0034647 A1
(43) Pub. Date: Feb. 1, 2024

(54) DEVICE FOR SEPARATING LEGIONELLA

(71) Applicant: Georg Fischer JRG AG, Sissach (CH)

(72) Inventors: Minh Hop NGUYEN, Haegendorf (CH); Steffen LEHMANN, Bad Saeckingen (DE); Alen PAVLIC, Obfelden (CH); Enrico CAMELIN, Gelterkinden (CH); Juerg DUAL, Elsau (CH); Stephan BUERLI, Wintersingen (CH); Lucas Raphael ROSENTHALER, Therwil (CH)

(21) Appl. No.: 18/223,295

(22) Filed: Jul. 18, 2023

(30) Foreign Application Priority Data
Jul. 26, 2022 (EP) 22 186 943.1

(51) Int. Cl. C02F 1/36; B01D 21/28; B06B 1/06

(52) U.S. Cl. CPC C02F 1/36; B01D 21/283; B06B 1/0622; C02F 2303/04

(57) ABSTRACT

A device for concentrating and separating Legionella and/or amoebas by acoustophoresis in tap water.

### CROSS-REFERENCE TO RELATED APPLICATION

This application claims the benefit and priority of European Patent Application No. 22 186 943.1 filed Jul. 26, 2022. The entire disclosure of the above application is incorporated herein by reference.

### BACKGROUND OF THE INVENTION

### Technical Field

The invention relates to a device for concentrating and separating Legionella and/or amoebas by acoustophoresis in tap water, preferably drinking water, including a flow chamber having an inlet opening through which the tap water flows in and an outlet opening through which the tap water flows out, wherein the openings are opposite to one another, and an opening for discharging concentrated Legionella and/or amoebas, wherein the flow chamber has dimensions, preferably a length, a height, and a width, and at least two transducers arranged outside the flow chamber, on two sides of the flow chamber in each case, for applying acoustic energy to the flow chamber for generating standing waves.

### DISCUSSION

Acoustophoresis is a method for concentrating and/or separating particles in a medium using the radiation pressure of intensive soundwaves. The particles can be, for example, dirt particles, bacteria, Legionella, or amoebas. Standing soundwaves, which exert forces on particles, can be induced for the generation of the radiation pressure. This method is also known in acoustofluidics and has been applied for several decades. The pressure profile of a standing wave varies between areas having high pressure or pressure difference and areas having low acoustic pressure, wherein the low area is to be found in the pressure nodes. Such standing waves are thus used to concentrate and/or separate particles in a medium.

WO2013/138797 discloses a method for separating contaminants in a host fluid, wherein the contaminants are particles of any size which are to be separated using the disclosed method. The ultrasonic waves are adapted in accordance with the particle sizes.

It is the aspect of the invention to propose a device which is designed for concentrating and separating Legionella and/or amoebas in tap water.

### SUMMARY

This aspect is achieved in that at least one of the dimensions of the flow chamber is designed in consideration of the acoustic contrast factor Φ of the Legionella and/or amoebas so as to generate one or more frequencies and pressure amplitudes of the standing waves such that the Legionella and/or amoebas concentrate or collect in the pressure nodes of the standing wave.

The acoustic contrast factor Φ is defined as follows:

$$Φ = \frac{1}{3} [ \frac{5 {ρ}_{p} - 2 {ρ}_{0}}{2 {ρ}_{p} + {ρ}_{0}} - \frac{{κ}_{p}}{{κ}_{0}} ]$$

and thus in dependence on the density ρ₀ and the compressibility K₀ of the tap water, and also in dependence on the density ρₚ and the compressibility Kₚ of the particle, that is to say that of the Legionella and the amoebas.

The device according to the preferred embodiment of the invention for concentrating and separating Legionella and/or amoebas by acoustophoresis in tap water includes at least two transducers, each arranged outside the flow chamber on two sides of the flow chamber, for applying acoustic energy to the flow chamber. The transducers are preferably each positioned in the same plane and also offset in relation to one another perpendicular to the flow direction on two sides of the flow chamber in each case. At least two standing soundwaves are thus achieved in the flow chamber, through which tap water flows.

An ultrasonic transducer, preferably having a piezoelectric element or preferably having a layered piezoelectric element, can be used as the transducer. However, drives/actuators, such as oscillating coils or eccentric drives, can also be used as transducers.

The device according to the preferred embodiment of the invention includes an opening for discharging the Legionella and/or amoebas. It is advantageous if a line is connected thereon, which enables the concentrated Legionella and/or amoebas to be discharged immediately.

The flow speed of the tap water having the concentrated Legionella and/or amoebas in the discharge line preferably corresponds to at least the flow speed in the flow chamber. A negative pressure is preferably present in the discharge line in comparison to the pressure in the flow chamber.

The flow chamber of the device according to the preferred embodiment of the invention has dimensions, preferably a length, a width, and a height, wherein at least one of the dimensions of the flow chamber is designed in consideration of the acoustic contrast factor Φ of the Legionella and/or amoebas so as to generate one or more frequencies and pressure amplitudes of the standing waves such that the Legionella and/or amoebas concentrate or accumulate in the pressure nodes of the standing wave.

The dimensions or one of these dimensions, such as height, width, and length of the flow chamber, are optimized in such a way that a minimum of energy has to be applied to concentrate and separate the Legionella and/or amoebas in the tap water.

It has proven to be advantageous if the height and the width are designed such that the pressure nodes, which arose in accordance with the standing waves generated by the transducer, are located in a line with the opening for discharging the Legionella and/or amoebas and preferably extend over at least a part of the length of the flow chamber. Legionella and/or amoebas can thus be easily separated, since they accumulate in the pressure nodes and are discharged in a line up to and then through the opening.

It has been shown to be a preferred embodiment if the flow chamber has a rectangular cross-sectional area.

It has proven to be advantageous that at least one transducer is arranged in each case at least on one side of the height and at least on one side of the width, in order to excite the flow chamber from two sides. The pressure fields of the generated standing waves are thus superimposed. The two waves can thus each have pressure nodes at different separation distances along the length of the flow chamber. The transducers are preferably each arranged in the middle of a side of the cross section.

The dimensions of the height and the width of the flow chamber are preferably designed in different lengths. In this way, the pressure fields, due to the transducers arranged on the sides, wherein one transducer is arranged on a side of the width and the other source is arranged on a side of the height, are superimposed and the Legionella and/or amoebas are also guided from the corners of the flow chamber into the middle or into the pressure nodes.

The transducers preferably generate an additional acoustic pressure or pressure difference in the flow chamber of at least 5 Mpa, preferably 10 Mpa or more. The environment of a resonance frequency of the flow chamber is preferably selected which is preferably constructed from a low-damping material. A minimum energy expenditure is thus required. A frequency between 15 kHz and 150kHz is preferably applied by the transducers for this purpose.

It is advantageous if the flow chamber has a height and a width by means of which the frequencies of a standing wave generated by the transducers are generated which have pressure nodes in the center of the cross section of the flow chamber.

It is advantageous over alternative separation methods such as ultrafiltration, chemical treatment, etc. to concentrate and separate the Legionella and amoebas without contact and noninvasively so as to prevent invasive interventions in a tap water system.

It has proven to be a preferred embodiment when the standing waves in the flow chamber are in the range of 15 kHz to 150 kHz, preferably 20 kHz to 60 kHz. The environment of a resonance frequency of the flow chamber is preferably selected, which is preferably constructed from a low-damping material.

It is advantageous if the length of the flow chamber is inversely proportional to the acoustic contrast factor Φ of the Legionella and/or amoebas. That is to say, the smaller the acoustic contrast factor Φ of the Legionella and/or amoebas, the longer the flow chamber has to be. Moreover, the required acoustic pressure in the flow chamber scales inversely proportional to the square root of the acoustic contrast factor Φ of the Legionella and/or amoebas. That is to say, the smaller the acoustic contrast factor Φ of the Legionella and/or amoebas, the greater the acoustic pressure in the flow chamber has to be in order to achieve the desired effect of accumulating the Legionella and/or amoebas at the pressure nodes.

The length, In dependence on the volume flow, the pressure amplitude, and the acoustic contrast factor Φ of the Legionella and/or amoebas, of the flow chamber is preferably 15 mm to 150 cm. It has been shown that over these distances, in dependence on frequencies, pressure amplitudes, and acoustic contrast factor Φ of the Legionella and/or amoebas, the Legionella and/or amoebas have enough time to accumulate in the pressure nodes.

It has proven to be advantageous “f the height of the flow chamber is at least 16 mm and the width is at least 16 mm. In addition to the adaptation to the acoustic contrast factor, the flow chamber is also connectable without problems to a pipeline having a diameter of 16 mm.

The flow chamber preferably has a variable wall thickness having depressions and thickenings from at least 1 mm to at most 10 mm along the flow chamber. The wall is thus not made planar, but rather has a structured surface.

The radii in the wall transitions to the inside of the structure of the flow chamber are at least 1/200 of the cross-sectional dimensions (B and H).

It has proven to be advantageous if the material of the flow chamber is produced from a material compatible with tap water, which moreover reflects acoustic waves and absorbs little, such as preferably copper alloys such as gunmetal, brass, or rustproof steels.

This wall thickness and also the mentioned materials enable good transmission of the acoustic energy from the transducer to the tap water and moreover also provides the flow chamber with sufficient stability to meet the requirements of water lines.

It is advantageous if the transducers are aligned perpendicularly to the longitudinal axis of the flow chamber and are arranged on the same plane and also on different planes along the longitudinal axis of the flow chamber. That is to say, the transducers can be arranged at different positions along the longitudinal axis and also at the same height along the longitudinal axis.

The transducers are preferably connected to a mass or the oscillating piston, which is in turn connected via a spring element, such as a rubber cushion, to the flow chamber, in order to thus guide the acoustic energy from the transducer into the tap water. The transducer is attached here in its action direction to the oscillating piston, this has contact with the tap water in the flow chamber and is connected via the spring element to the mechanical structure of the flow chamber. The width of the contact surface of the oscillating piston with the tap water is preferably at least 40% as wide as the inner cross-sectional width of the flow chamber perpendicular to the action direction of the respective transducer. It has also been shown to be a preferred embodiment if the length of the piston, in dependence on the mass and the length of the flow chamber, is at least 10 mm long. The oscillating piston consists of the same materials as those used in the flow chamber. The mass of the oscillating piston is, in accordance with the materials used in the flow chamber, as described in accordance with the geometry of the flow chamber. The spring-mass system consisting of transducer, oscillating piston, and spring element and without water contact preferably has a resonance frequency of 50 Hz to 150 kHz. It has proven to be advantageous that the spring element consists of a tap water-compatible material such as EPDM and is sealed off without dead space between oscillating piston and tap water. It preferably has a low damping between 0.5% and 30% and a modulus of elasticity between 30 MPa and 30 GPa.

The flow chamber of the device according to the invention includes an inlet opening and an outlet opening through which the tap water flows in and flows back out again. The openings are opposite to one another, preferably such that the flow chamber can be integrated in a pipeline. The device according to the invention is connected via the inlet and the outlet openings to the pipeline for the drinking water.

### DRAWINGS

An exemplary embodiment of the invention will be described on the basis of the figures, wherein the invention is not only restricted to the exemplary embodiment. In the figures:

FIG. 1 shows a schematic illustration of a device according to the invention,

FIG. 2 shows the cross-section of a flow chamber, and

FIG. 3 shows a spring-mass system for connecting the transducer to the flow chamber.

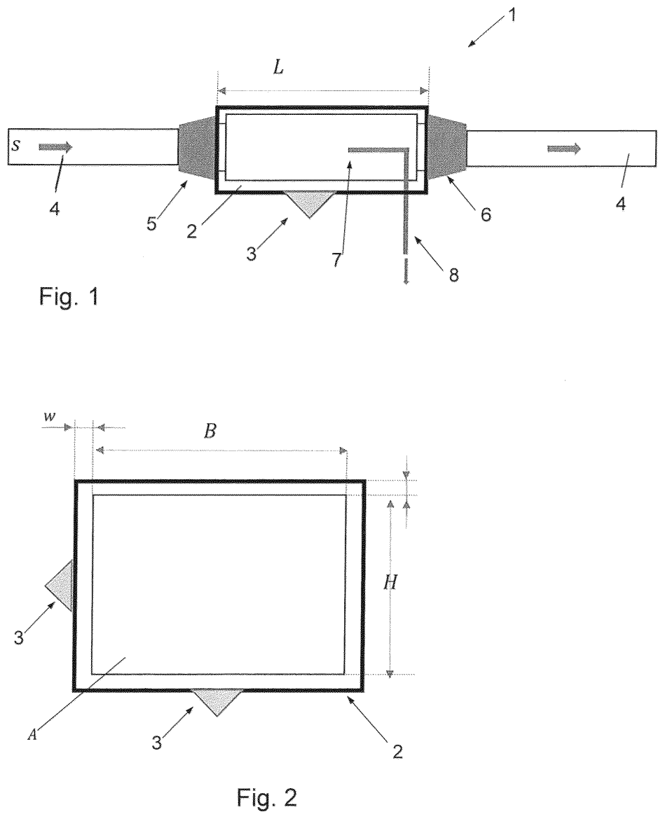

The drawing shown in FIG. 1 shows a schematic representation of a device 1 according to the invention. The device for concentrating and separating Legionella and/or amoebas by acoustophoresis is arranged in a water line 4. The device 1 includes a flow chamber 2, which is connected at each of the two end faces via inlet and outlet openings 5, 6 to the pipeline 4. The tap water flows through the flow chamber 2 accordingly. The transducers 3 for generating the acoustic energy are arranged on at least two sides of the flow chamber 2. Wherein one transducer 3 is arranged on a side of the width B and one transducer 3 is arranged on a side of the height H, respectively. Pressure field superpositions are achieved by the sound waves generated via the transducers 3 due to the rectangular cross section, as is apparent in FIG. 2, and because the height H and the width B have different lengths, the Legionella and/or amoebas are also carried or guided from the corners into the middle to the pressure nodes. The Legionella and/or amoebas collected in the pressure nodes are then guided via an opening 7 for discharging the Legionella and/or amoebas, which is also preferably on a line with the pressure nodes. A pipeline 8 for discharging the Legionella and/or amoebas preferably adjoins the opening 7. It has proven to be advantageous if the height H and the width B are designed in such a way that the acoustic pressure or pressure difference in the flow chamber 2 is at least 5 Mpa, preferably 10 Mpa and higher. Wherein the transducers 3.1 and 3.2 are preferably operated in the environment of a resonance frequency of the flow chamber, which is preferably constructed from a low-damping material, and thus the least possible energy consumption is applied. The sound waves preferably propagate at a frequency of 15 kHz to 150 kHz in the tap water. The pressure nodes, which extend at least over a part of the length L of the flow chamber 2, thus preferably form in the center of the cross section A. It is advantageous if at least an acoustic pressure or pressure difference of 0 MPa is present in the center, due to which the Legionella and/or amoebas accumulate there and are guided into the opening 7.

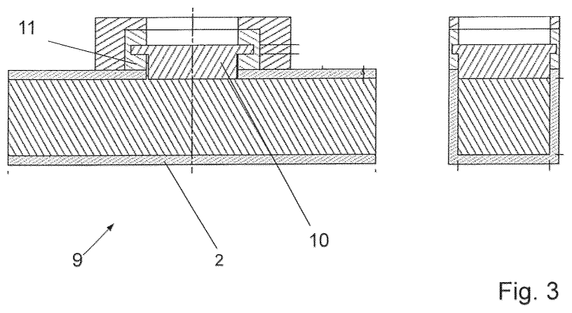

FIG. 3 depicts the spring-mass system 9, which represents the connection between the flow chamber 2 and the transducer 3. The oscillating piston preferably has a width of at least 40% of the width of the flow chamber, wherein the illustrated embodiment covers the complete width. The piston 10 therefore contacts the tap water located in the flow chamber. The oscillating piston 10 is mounted in a spring element 11, which is connected to the flow chamber 2.

What is claimed is:

1. A device (1) for concentrating and separating Legionella and/or amoebas by acoustophoresis in tap water (4), comprising a flow chamber (2) having an inlet opening (5) through which the tap water flows in and an outlet opening (6) through which the tap water flows out, wherein the openings (5, 6) are opposite to one another, and an opening (7) for discharging concentrated Legionella and/or amoebas, wherein the flow chamber (2) has dimensions, preferably a length (L), a height (H), and a width (B), and at least two transducers (3) arranged outside the flow chamber (2), on two sides of the flow chamber (2) in each case, for applying acoustic energy to the flow chamber (2) to generate standing waves, wherein at least one of the dimensions of the flow chamber is designed in consideration of the acoustic contrast factor Φ of the Legionella and/or amoebas so as to generate one or more frequencies and pressure amplitudes of the standing waves such that the Legionella and/or amoebas concentrate or accumulate in the pressure nodes of the standing waves.

2. A device (1) according to claim 1, wherein at least one of the dimensions of the flow chamber is designed in consideration of the acoustic contrast factor Φ of the Legionella and/or amoebas so as to generate one or more frequencies and pressure amplitudes of the standing waves such that the pressure nodes, the standing waves generated by the transducers, are in a line with the opening for discharging Legionella and/or amoebas.

3. A device according to claim 1, wherein the flow chamber has a rectangular cross-sectional area.

4. A device according to claim 1, wherein the height H and the width B of the flow chamber are of different lengths.

5. A device according to claim 1, wherein the height (H) and the width (B) of the flow chamber (2) are designed in such a way that by means of the generated standing waves, an acoustic pressure or pressure difference of at least MPa, preferably 10 MPa and higher is generated in the flow chamber (2), preferably at a generated frequency of 15 kHz to 150 kHz.

6. A device (1) according to claim 1, wherein at least one of the dimensions of the flow chamber is designed in consideration of the acoustic contrast factor Φ of the Legionella and/or amoebas so as to generate one or more frequencies and pressure amplitudes of the standing waves which have a minimum acoustic pressure or pressure difference in the center of the cross section (A) of the flow chamber (2) of 0 MPa.

7. A device (1) according to claim 1, wherein the standing waves in the flow chamber (2) are in the range of 15 kHz to 150 kHz, preferably in the environment of a resonance frequency of the flow chamber, which is preferably constructed from a low-damping material, and thus requires the least possible energy consumption.

8. A device (1) according to claim 1, wherein the maximum acoustic pressure or pressure difference of the flow chamber (2) is inversely proportional to the square root of the acoustic contrast factor Φ of the Legionella and/or amoebas.

9. A device (1) according to claim 1, wherein the length (L) of the flow chamber is 15 mm to 150 cm.

10. A device (1) according to claim 1, wherein the height (H) of the flow chamber is at least 16 mm and the width (B) is at least 16 mm.

11. A device (1) according to claim 1, wherein the flow chamber (2) has a variable wall thickness (w) having depressions and material thickenings of at least 1 mm and at most 10 mm.

12. A device (1) according to claim 1, wherein the flow chamber (2) is produced from a material compatible with tap water, which moreover reflects acoustic waves and absorbs little, such as preferably copper alloys, especially preferably gunmetal, brass, or rustproof steels.

13. A device (1) according to claim 1, wherein the transducers are aligned perpendicular to the longitudinal axis of the flow chamber and are arranged on the same plane and also on different planes along the longitudinal axis of the flow chamber (2).

14. A device (1) according to claim 1, wherein the transducers (3) are formed from a piezoelectric element or a layered piezoelectric element.

15. A device (1) according to claim 1, wherein the transducers are connected to a mass or an oscillating piston (10), wherein the oscillating piston (10) is connected via a spring element (11) to the flow chamber (2).
